(19) United States
(12) Patent Application Publication
(10) Pub. No.: US 2021/0371483 A1
(43) Pub. Date: Dec. 2, 2021

(54) Composition for Inactivating Spores by Means of Antimicrobial Peptides

(71) Applicant: Robert Bosch Gesellschaft Fur Medizinische Fruschung mbH, Stuttgart (DE)

(72) Inventors: Sabine Nuding, Ebersbach (DE); Eduard Stange, Stuttgart (DE)

(21) Appl. No.: 16/343,609

(22) PCT Filed: Oct. 6, 2017
(86) PCT No.: PCT/EP17/75432

§ 371 Date: Apr. 19, 2019

(30) Foreign Application Priority Data
Nov. 3, 2016 (DE) 10 2016 221 559.0

(51) Int. Cl. C07K 14/47; A61L 2/00

(52) U.S. Cl. CPC C07K 14/4723; A61K 38/00; A61L 2/0088

(57) ABSTRACT

A composition, which comprises at least one natural, recombinant, or synthetic human antimicrobial peptide, is selected from a human defensin or cathelicidin or functional fragments or combinations thereof, for inactivating bacterial spores.

The present invention relates to the use of a composition of antimicrobial peptides for inactivating bacterial spores, more particularly its use as disinfectant and its use as medicament for preventing a recurrence in the case of infections.

### PRIOR ART

Clostridium difficile is the most common pathogen in nosocomial and antibiotics-associated diarrheas. This means that C. difficile infections (CID) in particular represent an increasingly important problem in healthcare; they frequently occur during or after an antibiotic therapy. For example, if a substantial portion of the natural gut flora is destroyed as a result of administration of a broad-spectrum antibiotic, it is possible for C. difficile to greatly multiply owing to its particular resistance properties and the lack of competition.

Transmission in the nosocomial environment is primarily caused by the uptake of spores, which may have been acquired from other patients directly, via the hands of caregivers or indirectly from inanimate objects. After subsidence of the first infection, a recurrence additionally occurs in approx. 20% of patients. Also the cause here are the spores which are not attacked by the antibiotics during the therapy. Moreover, the C. difficile spores cannot be sufficiently inactivated by the customary (including alcoholic) disinfectants. They can persist for months in the environment on furnishings and medical instruments and later germinate again. They are therefore the main cause of recurrences in C. difficile infections.

Currently, suitable agents are not available in the prior art against the spore-initiated reoccurrence of C. difficile infections and against the associated resistances to antibiotics that occur.

Spores are endurance forms which can remain capable of multiplication for many decades and are particularly resistant to the environment. In contrast to their vegetative cells, they are particularly resistant to dehydration and irradiation, but also to a range of chemicals.

The formation of spores is also called sporulation. There is increased spore formation after nutrient consumption when the growth conditions for the spore-forming bacteria become unfavorable. When the external conditions become favorable again and certain chemical signals (e.g., glucose, adenosine, amino acids) act on the spores, spores can then germinate within a short time and initiate the formation of a bacterial population. Specifically germination represents a major problem in the case of pathogenic bacteria, since they, in their vegetative-cell form, can remultiply and produce toxins again.

Thus, there is not only an increased need for novel antibiotics, especially against the hitherto resistant bacteria, but also a need for novel treatment methods which also act specifically on spores.

### DISCLOSURE OF THE INVENTION

Against this background, it is an object of the present invention to provide a composition which reduces or completely avoids the aforementioned disadvantages from the prior art.

According to the invention, this object and other goals are achieved by the use of a composition comprising at least one natural, recombinant or synthetic human antimicrobial peptide selected from a human defensin or cathelicidin, or functional fragments or combinations thereof, for inactivating bacterial spores.

Furthermore, the goal is achieved by the composition comprising at least one natural, recombinant or synthetic human antimicrobial peptide selected from a human defensin or cathelicidin, or functional fragments or combinations thereof, for use in the inactivation of bacterial spores.

Here and in general, a “human antimicrobial peptide” is understood to mean an antimicrobially effective peptide of human origin, or which is derived from such antimicrobially effective peptides of human origin or is prepared, synthesized or produced in some other way and has the structure and function of such antimicrobially effective peptides of human origin.

“Defensins” are, in the present invention and in general in the specialist field, to be understood to mean a family of highly effective antimicrobial peptides. They are important key molecules of innate immunity which protect humans against microorganisms and which additionally form the composition of the microbiota of mucous membranes. It is known that defensins are active against bacteria, fungi and viruses. However, what is completely new is their destructive effect with respect to bacterial spores, as the inventors have been able to demonstrate.

In the prior art, it is currently known that humans produce two types of defensin, which are classified into alpha-defensins and beta-defensins on the basis of their sequence homology and the cysteine residues. Currently, six alpha-defensins and four beta-defensins have been characterized in humans, and 31 defensin genes have been identified. Whereas the beta-defensins are expressed at multiple locations in the gastrointestinal tract, the alpha-defensins are predominantly expressed in the small intestine. Mature human defensins contain between 30 and 40 amino acid residues, and each defensin has three disulfide bonds. Among the six human alpha-defensins, four are expressed in neutrophils and two are primarily expressed in Paneth cells, which are secretory epithelial cells and the main source of antimicrobial peptides in the small intestine.

Defensins can be produced chemically or else in genetically modified microorganisms and can be introduced into the colon using an appropriate dosage form for release in the terminal ileum without prior degradation or absorption in the small intestine.

“A cathelicidin” is understood here to mean the peptide cathelicidin as well as cathelicidin-related antimicrobial peptides, which are a family of polypeptides found in lysosomes of macrophages and polymorphonuclear leukocytes (PMNs). Cathelicidins play a critical role in the mammalian innate immune defense against invasive bacterial infections. Members of the cathelicidin family of antimicrobial polypeptides are characterized by a highly conserved region (cathelin domain) and a highly variable cathelicidin peptide domain. Cathelicidins were originally found in neutrophils, but have since then been found in many other cells, including epithelial cells and macrophages after activation by bacteria, viruses, fungi or the hormone 1,25-D, which is the hormonally active form of vitamin D. Cathelicidins are, in terms of size, within the range from 12 to 80 amino acid residues and have a wide variety of different structures. Most cathelicidins are linear peptides having 23 to 37 amino acid residues and fold to form amphipathic alpha-helices.

Accordingly, what is also true is that suitable antimicrobial peptides which can be inventive in connection with the present invention are not only the complete human antimicrobial peptide selected from human defensins or human cathelicidins, but also fragments thereof that still have the antimicrobial activity, with respect to bacterial spores, and function of the human antimicrobial peptide(s) used in the particular case. A person skilled in the art is, with the present invention, aware of means and methods for discovering suitable fragments of complete human antimicrobial peptides, especially due to C- or N-terminal truncation of the complete human antimicrobial peptide, and for testing their antimicrobial efficacy with respect to spores.

Similarly, a “functional fragment” of a human antimicrobial peptide is understood here and in general to mean a fragment of said peptides that still has a substantial antimicrobial activity with respect to bacterial spores, which activity is roughly comparable with that of the corresponding nonfragmented peptide.

In the present invention, it is in each case possible for not only one human antimicrobial peptide, but also a combination of, in each case, two or more, to be used according to the invention.

Here and in general, the expression “natural” covers any peptide which has been taken from a starting material where the peptide is found/produced naturally; thus, a natural peptide is, in general, isolated from its starting material, “isolated” meaning altered from its natural state “by the human hand”, i.e., if it occurs in nature, it has been taken or removed from its natural environment, or both. For example, a polynucleotide or a polypeptide which is naturally present in a living organism is not “isolated”; the same polynucleotide or polypeptide which is separated from the simultaneously occurring materials of its natural state is, however, “isolated” in the present sense. Similarly, what is meant in the present context by a “synthetic” sequence is any sequence which has been produced synthetically and has not been directly isolated from a natural starting material. “Recombinant” means gene technology-modified DNA which has been prepared by transplanting or splicing genes from one species into cells of a host organism of another species. Such DNA becomes part of the genetic material of the host and is replicated. Any protein/peptide expressed from such DNA can subsequently be isolated from the host cell and be used according to the invention.

For the isolation of naturally occurring human defensins or cathelicidin and for the preparation of recombinantly or synthetically prepared defensins or cathelicidin, a person skilled in the art is aware of adequate options in the prior art that are all part of his/her ability and knowledge and that represent customary procedures in the particular field.

The human antimicrobial peptide according to the invention can be prepared by means of DNA recombination technology using techniques well-known to specialists. It is possible to make use of methods well-known to a person skilled in the art in order to construct expression vectors which contain the sequences coding for a peptide of interest and corresponding transcription/translation control signals. Said methods include, for example, in vitro DNA recombination techniques, synthesis techniques and in vivo genetic recombination; see, for example, the techniques described in Sambrook et al., Molecular Cloning: A Laboratory Manual, 2nd edition. Cold Spring Harbor Laboratory Press, Cold Spring Harbor, N. Y. (1989).

Also known to specialists are many different synthesis methods for producing the human antimicrobial peptides, which include, for example, liquid-phase synthesis, solid-phase synthesis, Fmoc protection, BOC protection, etc., and, in this regard, a multiplicity of literature references is available and also many companies produce peptides to order, such as Sigma Aldrich for example. Reference can, for example, be made to Kent, “Chemical Synthesis of Proteins and Peptides”, (1988) Ann. Rev. Biochem. 57: 957-989, and Albericio and Martin, “Solid Supports for the Synthesis of Peptides”, (2008) supplement to Chimica Oggi/CHEMISTRY TODAY, 26: 29-34.

“Inactivating bacterial spores” is understood in general to mean the inhibition and the delaying of the germination of bacterial spores or the inhibition of the steady growth of newly formed vegetative cells. The inventors were able to demonstrate strikingly that the treatment of spores with human antimicrobial peptides results merely in a low degree of germination of the spores to form vegetative cells. This result was completely surprising, since antimicrobial peptides are known to act antimicrobially with respect to bacteria. A penetration of antimicrobial peptides into the spores and a possible destruction of the spores was hitherto unknown, especially since bacteria greatly differ in terms of structure from their spores.

According to one exemplary embodiment, the human antimicrobial peptides are those which are disclosed in patent application DE 10 2014 213 654 A1 and which can be used according to the invention. The content of said application is incorporated in the present application by reference.

According to one aspect of the invention, the spores are of aerobic or anaerobic bacteria.

Here and in general, the term “anaerobic” or “anaerobically” living “bacteria” means any bacterial organism which does not require oxygen for its growth. If oxygen is present, it may react negatively and may even die. Anaerobic bacteria can therefore be classified into obligate anaerobes, which are damaged by the presence of oxygen, aerotolerant organisms, which cannot use oxygen for growth, but tolerate its presence, and facultative anaerobes, which can grow without oxygen, but utilize oxygen when it is available.

The term “aerobic bacteria” by contrast means any bacterial organism which requires oxygen for its growth; they are also referred to as aerobes. Among the aerobes, a distinction is made between obligate aerobes and facultative aerobes. Obligate aerobes absolutely require oxygen for their respiration metabolism. Facultative aerobes are also referred to as aerotolerant or micro-aerotolerant. They can be cultured in the presence of oxygen without being damaged, since they are protected from the toxic effect of the oxygen by particular enzymes (e.g., superoxide dismutase). This has the advantage that the composition can be used against a multiplicity of bacterial spores, especially those which originate from pathogenic bacteria.

According to a further aspect of the invention, the spores are derived from bacteria selected from at least one of the following: Clostridium sporogenes, Clostridium difficile, Clostridium tetani, Clostridium botulinum, Clostridium perfringens, Clostridium novyi, Clostridium histolyticum, Clostridium sordellii, Clostridium ramosum, Clostridium innocuum, Clostridium septicum or Bacillus subtilis, Bacillus anthracis, Bacillus cereus, Bacillus licheniformis, Bacillus circulans, Bacillus coagulans.

Clostridium is a Gram-positive bacterial genus which belongs to the Firmicutes. What are concerned are obligate anaerobes capable of producing endospores. The individual cells are rod-shaped, this giving them their name, which comes from the Greek word “kloster” or “spindle”.

Bacillus is also a Gram-positive bacterial genus which belongs to the Firmicutes. The genus Bacillus is distinguished by the formation of endospores and an aerobic or facultatively aerobic growth. They multiply only under aerobic conditions and form lecithinase and catalase in some circumstances.

In a preferred embodiment of the invention, the composition is used for the inhibition of the germination of bacterial spores to form vegetative cells or of the steady growth thereof.

“Inhibition of the germination” is understood in the present invention to mean that most of the spores remain in their endurance forms and do not germinate to form vegetative cells. The endurance form corresponds to the spore form that is incapable of growth and nonpathogenic. The size of the proportion remaining in the spore form is determined, inter alia, by the nature of the composition, the concentration of the composition and the nature of the bacterial spores. Furthermore, the spore can be destroyed by a treatment with the inventive composition itself, i.e., be completely inactivated.

Here and in general, a “germination” is understood to mean a triphasic mechanism of a bacterial spore in the direction of a vegetative cell. In a first step, the spore is activated. The activation essentially consists in an increase in the permeability of the spore shell, with the result that spore germination-inducing substances can penetrate from the outside. This can take place owing to aging or owing to mechanical damage. If certain species-specific inducers are present in the external environment after this activation phase, germination in the narrower is initiated. Substances acting as inducers of the germination are those which are generally present in the natural environment under conditions favorable for the growth of the bacterium. Examples thereof are energy sources (e.g., glucose) and nutrients (e.g., adenosine and L-alanine) that are utilizable by the bacterium. During the germination, the spore shell is weakened by partial degradation. During outgrowth, the cell increases in volume as a result of water uptake and growth (formation of new cellular components), bursts the spore shell and forms a new cell wall. In the context of this invention, the composition can intervene effectively in each germination phase and can inactivate the bacterial spore or prevent it from germinating.

In a further preferred embodiment of the invention, the human defensin or cathelicidin is selected from the group comprising human beta-defensin-3 (hBD-3), cathelicidin LL-37, or fragments or combinations of one or more thereof.

Human beta-defensin 3 (hBD3) is a protein composed of 45 amino acids that is the most cationic defensin with a net charge of +11. hBD3 is present as dimer in solution and, under physiological salt conditions, exhibits in the micromolar concentration range a good antimicrobial activity against Gram-negative bacteria and, unlike the alpha-defensins and hBD1 and 2, against Gram-positive bacteria, including methicillin-resistant Staphylococcus aureus (MRSA) and vancomycin-resistant Enterococcus faecium strains. To date, hardly anything is known about the molecular mechanisms of the mode of action of hBD3. However, in a treatment of Staphylococcus aureus cells with hBD3, it was possible to observe the appearance of signs of perforation of the cell well, evaginations of the plasma membrane and subsequent cell lysis, similar to what has been observed in penicillin-treated cells.

LL-37 is a human antimicrobial peptide from the group of the cathelicidins. It is mainly produced in immune cells and is part of the innate immune response and also of the apoptosis of body-endogenous cells. It is a transport protein, the incorporation of which into the cell wall of Gram-positive bacteria, on the one hand, and into the cell membrane, on the other, leads to a loss of ions and small molecules. It is encoded by the CAMP gene. The production of LL-37 is induced especially by stimulation of TLR-9, but also TLR-2 and TLR-4, and indirectly by vitamin D.

Since the effect of hBD3 and LL37 does not extend just to bacteria, but is also directed at bacterial spores, a completely new form of therapy is provided that makes it possible for a recurrence to be prevented in advance in a prophylactic manner, during a bacterial disease and after a bacterial disease.

According to a preferred embodiment, the composition is used as disinfectant, especially preferably as disinfectant of material surfaces.

Here and in general, “disinfectant” is understood to mean a composition which can be utilized for the microbial reduction of microbes capable of multiplication. Furthermore, disinfectants are also understood to mean those compositions having sporicidal effects, i.e., are also active against spores (microbes incapable of multiplication). This type of disinfectant is also called sporicide. As a result, it is possible to prevent especially a recurrence initiated by germinating spores.

Disinfectants of material surfaces are to be understood here to mean surface disinfectants, i.e., an agent which is preferably suited to the disinfection of surfaces of objects and relatively large areas such as, for example, floors or the like.

In a further preferred embodiment, the composition is used for disinfection in mammals, especially humans, especially preferably on the human skin, preferably on hands, mucous membrane or a wound.

In this case, the composition can be used in terms of hand disinfection, of wound and mucous-membrane disinfectant or of skin antiseptic. The disinfectant can be used both for therapeutic purposes and for everyday use. This offers the advantage of a comprehensive protection with respect to bacteria which may act pathogenically owing to germination.

According to a further aspect of the invention, the composition is used as medicament for preventing a recurrence in the case of infections initiated by bacterial spores.

According to a further aspect of the invention, the composition is used in the therapy or prophylaxis of an infection, especially of a recurrence, initiated by bacterial spores.

In addition to use as disinfectant, the composition can also be used as medicament. In this case, the composition can be used effectively during bacterial diseases, and for preventing a recurrence in the case of diseases which have subsided. Furthermore, the composition can also be used as prophylaxis.

Accordingly, the present invention provides pharmaceutically safe compositions comprising a therapeutically effective amount of one or more of the herein-described compounds and/or compositions in a common formulation with one or more pharmaceutically safe carriers (additives) and/or diluents. Examples of administration include parenteral administration, for example intravenous, intradermal, subcutaneous, transdermal, transmucosal administration. The determination of the amounts and the dosage forms and also of the routes of administration that are suitable in each case is part of the ability and the knowledge of a person skilled in the art. They may vary with respect to the patient, the particular disease and in consideration of other factors. The amount of active ingredient that can be combined with a carrier material in order to produce an individual dosage form will generally be that amount of the compound and/or the composition that leads to a therapeutic effect. In general, said amount will be within the range from about 1% to about 99% active ingredient, preferably from about 5% to about 70%, most preferably from about 10% to about 30%. The actual dosage levels of the active ingredients in pharmaceutical compositions of this invention can vary, meaning that the amount of active ingredient that is arrived at is the amount which is effective for achieving the desired therapeutic response in the case of a particular patient, a particular composition and a particular route of administration without being toxic to the patient.

The selected dosage level will depend on various factors, including the activity of the particular pharmaceutical compositions of the present invention that are used or of the ester, salt or amide thereof, the route of administration, the time of administration, the excretion rate or the metabolism of the particular pharmaceutical compositions used, the length of treatment, other medicaments, compounds and/or compositions and/or materials used in combination with the particular pharmaceutical compositions used, the age, the sex, the weight, the state, the general health and the anamnesis of the treated patient, and also similar factors well-known in the field of medicine. It is possible to use a daily, weekly or monthly dosage (or a different time interval).

The expression “pharmaceutically safe”, as used in the present text, relates to those compounds, materials, compositions and/or dosage forms which, in the context of a solid medical assessment, are suitable for use in contact with the tissues of humans and animals without severe toxicity, irritation, allergic reaction or other problems or complications according to an appropriate risk-benefit ratio.

The expression “pharmaceutically safe carrier” means, in the present context, a pharmaceutically safe material, a pharmaceutically safe composition or a pharmaceutically safe base substance, such as a liquid or solid filler, a diluent/extender, an excipient or a solvent-encapsulating material, which is involved the transfer or transport of the present compound and/or composition from one organ or body part to another organ or body part. Every carrier must be “safe” in the sense that it is compatible with the other constituents of the formulation and is not harmful to the patient.

Pharmaceutically safe carriers and excipients with optionally further additives are generally known in the prior art and are, for example, described in the work by Kibbe A., Handbook of Pharmaceutical Excipients, third edition, American Pharmaceutical Association and Pharmaceutical Press 2000. According to the invention, additives encompass any compound or composition which is advantageous for the therapeutic use of the composition, including salts, binders, solvents, dispersants and other substances customarily used in connection with the formulation of medical products.

The expression “therapeutically effective amount” means, in the present context, that amount of a compound and/or a composition, of a material or of a composition comprising a pharmaceutical composition of the present invention which is effective for eliciting a certain desired therapeutic effect in at least a subpopulation of cells in an animal, specifically in accordance with a risk-benefit ratio appropriate for every medical treatment.

### Advantages of the Invention

The use of the composition in the context of the invention offers the advantage of providing a composition suitable for inactivating bacterial spores. Furthermore, the invention offers the advantage that the composition is active against a multiplicity of bacterial spores, especially spores of pathogenic bacteria, so that an inhibition of germination may possibly lead to a prevention of a recurrence.

Furthermore, the pharmaceutical composition can be used both as medicament and as disinfectant. This offers the advantage of a complete therapy within a bacterial disease, but also as prevention within a prophylaxis or as additional therapy following a bacterial disease. The composition according to the invention can, for example, be used in a supporting manner during an antibiotic therapy.

The present invention is, in terms of its use, not restricted to the details of the establishment and arrangement of the components that are presented in the description or illustrated in the drawings. The invention allows other embodiments and can be carried out in various ways. In addition, the manner of expression and terminology that are used here serve for descriptive purposes and are not to be considered to have a limiting effect. In the present text, the use of “inclusive of”, “comprising” or “having”, “containing”, “including” and variations thereof is intended to encompass what is listed afterwards as well as equivalents thereof and what is additional.

The present invention is further illustrated by the following examples, which are in no way to be understood as having a further limiting effect. The entire content of all citations in the present application (inclusive of literature references, granted patents, published patent applications and codependent patent applications) is hereby expressly incorporated in the present text by reference. In the event of any conflict, the present description, inclusive of any definitions herein, takes precedence.

The figures are described below.

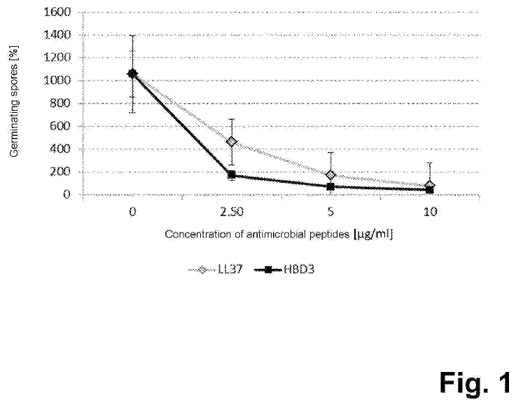

FIG. 1 shows the results of a treatment of a suspension of vegetative cells and spores after a heat-treatment to kill the vegetative cells with the exemplary antimicrobial peptides LL37 and hBD3 as a function of the concentration of the antimicrobial peptide; and

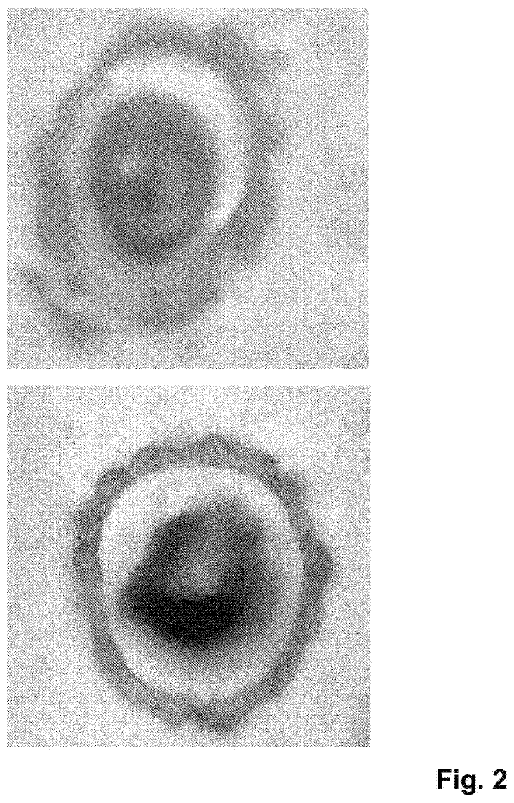

FIG. 2 shows an electron microscopy of C. difficile spores which had been incubated with gold-labeled HBD3 peptides. HBD3 was able to penetrate into the spores (black particles); and

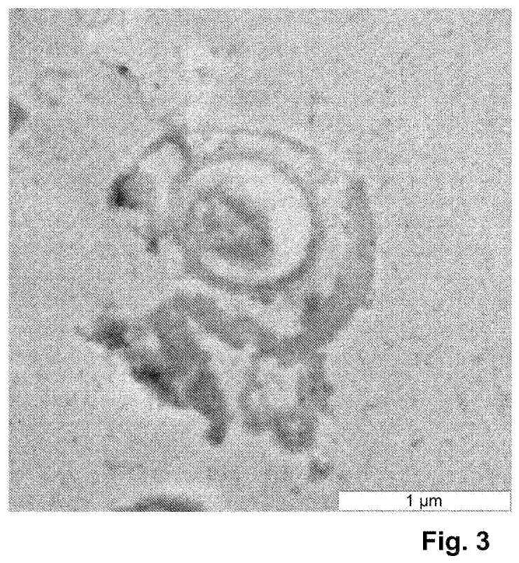

FIG. 3 shows an electron microscopy of C. difficile spores. In this C. difficile strain, the spores were destroyed structurally by HBD3.

### EXAMPLES

### Embodiments of the Invention

Effect of Antimicrobial Peptides on C. difficile Spores

The effects of antimicrobial peptides on C. difficile spores were investigated by first enriching a bacterial suspension with spores so that at least 70% spores were present. The spore suspension was incubated for 60 minutes with various defensins (HBD1, HBD2, HBD3) and the cathelicidin LL37 (recombinant defensins: Peptanova Sandhausen, HBD1 order No. 4337; HBD2; 4338, HBD3 4382/recombinant LL37: Innovagen Lund Sweden, order No. SP-LL-37-1). Thereafter, aliquots of the samples were plated out on Columbia blood agar and incubated anaerobically for at least 4 days. The germinated spores in an untreated control and the preparations containing antimicrobial peptides were counted.

It became apparent that, in the case of the samples which were incubated with HBD3 or LL37, significantly fewer germinated spores were present in comparison with the controls.

In order to able to establish with certainty whether either germinating bacteria are rapidly killed by the presence of antimicrobial peptides in the medium or the spores are in fact prevented from germinating, the experiment was repeated, but at the end of the incubation time, the peptides were washed out and the medium was free of peptides.

Despite the wash-out, the inhibitory effect of HBD3 and LL37 was nevertheless surprisingly maintained, meaning that it was also possible to demonstrate in these experiments that both defensins and cathelicidins are effective against spores.

In further experiments, the inventers wanted to make the unambiguous assignment that resultant bacterial colonies can be attributed with certainty to the germinated spores and not to the steady growth of residual vegetative cells. To this end, incubation and wash-out were followed by killing the residual vegetative cells using heat (61° C., 30 min). This heat treatment kills only the vegetative cells, but not the spores.

These experiments revealed a concentration-dependent decrease in germinating spores for the two effective antimicrobial peptides, with HBD3 already showing in lower concentrations a somewhat higher efficacy. The results of said experiments are summarized in FIG. 1. There, the different effects of LL37 in comparison with HBD3 on germinating spores are reproduced in a graph.

It is clear that just a low concentration (2.5 μg/mL) of HBD3 is sufficient for greatly lowering the number of germinating spores. Whereas there is still a distinct difference between the effect of LL37 and HBD3 at low concentrations, said difference is hardly noticeable at larger concentrations of antimicrobial peptides (10 μg/mL).

In further experiments with gold-labeled HBD3 peptides, it was possible to demonstrate that HBD3 penetrates into the exosporium, the outer wall of the spores, and destroys parts of the spore structure. These results are depicted strikingly in FIGS. 2 and 3.

To date, it was only known that antimicrobial peptides, especially the defensin HBD3 and the cathelicidin LL37, kill vegetative cells effectively and that the effect can moreover be intensified in conjunction with antibiotics. What is completely new by contrast is the finding that spores as well can be destroyed by the treatment with antimicrobial peptides.

Furthermore, electron micrographs confirm the penetration of HBD3 directly into the spores. According to the current prior art, there is no known antibiotic or peptide which would be able to achieve this.

The best known mechanism of action of antimicrobial peptides is pore formation in the bacterial membrane. What is completely new and surprising is that said mechanism of action can also be applied to the spore wall. Since the cell wall structure of bacteria greatly differs compared to the cell wall structure of bacterial spores, for example antibiotics which have one mode of action within the cell membrane are completely ineffective against spores, it was not possible to assume that human antimicrobial peptides are necessarily active against bacterial spores.

1. The use of a composition, comprising:
selecting at least one natural, recombinant, or synthetic human antimicrobial peptide from a human defensin or cathelicidin, or functional fragments or combinations thereof, for inactivating bacterial spores.

2. The use as claimed in claim 1, wherein the spores are of aerobic or anaerobic bacteria.

3. The use as claimed in claim 1, wherein the bacterial spores are derived from bacteria selected from at least one of the following: Clostridium sporogenes, Clostridium difficile, Clostridium tetani, Clostridium botulinum, Clostridium perfringens, Clostridium novyi, Clostridium histolyticum, Clostridium sordellii, Clostridium ramosum, Clostridium innocuum, Clostridium septicum or Bacillus subtilis, Bacillus anthracis, Bacillus cereus, Bacillus licheniformis, Bacillus circulans, and Bacillus coagulans.

4. The use as claimed in claim 1, further comprising:
using the composition for an inhibition of a germination of the bacterial spores to form vegetative cells.

5. The use as claimed in claim 1, further comprising:
selecting the human defensin or cathelicidin from a group comprising human beta-defensin-3, cathelicidin LL-37, or fragments or combinations of one or more thereof.

6. The use as claimed in claim 1, further comprising:
using the composition as a disinfectant.

7. The use as claimed in claim 1, further comprising:
using the composition as a disinfectant of material surfaces.

8. The use as claimed in claim 1, further comprising:
using the composition for disinfection in mammals including humans.

9. The use as claimed in claim 8, further comprising:
using the composition on human hands, a mucous membrane, or a wound.

10. The use as claimed in claim 1, further comprising:
using the composition as medicament for preventing a recurrence in a case of infections initiated by the bacterial spores.

11. A composition comprising:
at least one natural, recombinant, or synthetic human antimicrobial peptide selected from a human defensin or cathelicidin, or functional fragments or combinations thereof,
wherein the composition is used in an inactivation of bacterial spores.

12. The composition as claimed in claim 11, wherein the composition is used as disinfectant or medicament.

13. The composition as claimed in claim 11, wherein the composition is used in therapy or prophylaxis of an infection initiated by the bacterial spores.
